(19) United States
(12) Patent Application Publication
(10) Pub. No.: US 2023/0172652 A1
(43) Pub. Date: Jun. 8, 2023

(54) ELECTROSURGICAL GENERATOR WITH A LEAKAGE CURRENT DETECTION

(71) Applicant: OLYMPUS WINTER & IBE GMBH, Hamburg (DE)

(72) Inventors: Daniel RAMIN, Nuthetal (DE); Thomas FÄHSING, Berlin (DE); Jelle DIJKSTRA, Berlin (DE)

(73) Assignee: OLYMPUS WINTER & IBE GMBH, Hamburg (DE)

(21) Appl. No.: 18/077,001

(22) Filed: Dec. 7, 2022

Related U.S. Application Data
(60) Provisional application No. 63/287,004, filed on Dec. 7, 2021.

(51) Int. Cl. A61B 18/12; G01R 31/52; G01R 19/10; G01R 15/04; A61B 18/16

(52) U.S. Cl. CPC A61B 18/1206; G01R 31/52; G01R 19/10; G01R 15/04; A61B 18/16; A61B 2018/00892

(57) ABSTRACT

An electrosurgical generator which outputs high-frequency AC voltage for an electrosurgical instrument includes a leakage current detecting device for the connected electrosurgical instrument. The leakage current detecting device is embodied as a voltage measuring device, the inputs of which are connected in each case via a capacitive coupling to an active and a neutral line of the output line and which has a bipolar voltage divider having a predetermined fixed ratio. An asymmetry detector connected via a capacitive coupling compares upper and lower voltage at the voltage divider, and outputs a fault signal for leakage current in the case of deviation of the ratio of upper to lower voltage from the predetermined fixed ratio.

The invention relates to an electrosurgical generator configured to output a high-frequency AC voltage to an electrosurgical instrument. Said electrosurgical generator comprises an inverter for high voltage, which generates the high-frequency AC voltage that is passed via an output line to an output for connection of the electrosurgical instrument, and a leakage current detecting device for the electrosurgical instrument connected to the output.

In electrosurgery or radio frequency surgery an electrosurgical instrument, such as an electric scalpel, is used to input high-frequency AC voltage into tissue of the human body in particular in order to cut or slice through the tissue by way of the heating thus caused. One advantage of this is that, at the same time as the cutting, it is also possible to stem bleeding by closing the affected vessels, and electrosurgical instruments are conceivable for further types of applications, such as for coagulation, for example.

Considerable powers are required for this purpose, specifically at frequencies of 200 kHz or higher up to 4000 kHz, typically around 400 kHz. Body tissue behaves like an ohmic resistance at such frequencies. The specific resistivity is greatly dependent on the type of tissue, however, and so the specific resistivities of muscles, fat or bones differ greatly from one another, specifically by up to a factor of 1000. This has the effect that, during operation, the load impedance of the electric scalpel may change rapidly and greatly depending on the tissue to be cut, proceeding from virtually infinity as the instrument approaches the tissue through to a virtual short circuit. That places special and unique demands on the electrosurgical generator, and in particular the high-voltage provision therefor, which do not arise in this way in other technological fields.

In order to satisfy these unique requirements, electrosurgical generators are typically constructed such that they have an inverter for the supply of the electrosurgical instrument, to which rectified current is fed. The inverter is typically embodied as a free-running single-ended generator having an LC resonant circuit. This construction has proved to be worthwhile. Furthermore, the applicant has developed a design of electrosurgical generators which provide a multilevel inverter as inverter. Frequency, amplitude and waveform of the AC voltage generated can thus be adjusted substantially freely. The high voltages required are achieved by means of an output transformer.

In view of the high output voltage that can be output by electrosurgical generators, protecting the patient is of particular importance. This includes controlled passage of the current flow on the patient, specifically from one electrode of the electrosurgical generator to the other. In this case, the electrode connected to the electrosurgical instrument is typically referred to as active electrode. Depending on the embodiment, the other electrode may likewise be connected to the instrument or, via a surface contact electrode, the patient in order thus to close the electric circuit. It is important for this last to occur completely and for no leakage currents to arise. The latter may become dangerous for the patient.

For safe operation of electrosurgical generators, it is therefore important for the occurrence of leakage currents to be detected rapidly and reliably. For this purpose, it is known to provide current sensors at the output connection of the electrosurgical generator. These sensors should preferably be arranged in direct proximity to the connection in order to avoid disturbing influences as a result of the internal construction of the generator. It is typical practice in the case of known electrosurgical generators, for the purpose of current measurement, to carry out a kind of fault current measurement that ascertains the difference between the current flowing away to the active electrode and the current returning via the surface electrode. Current sensors (current transformers) which operate according to the transformer principle are known for this purpose. Disadvantages of this arrangement are that the sensor system is complex and has specific structural space requirements, since the sensor system typically has to be arranged in direct proximity to the output in order to avoid incorrect measurements.

The invention addresses the problem of providing an improved electrosurgical generator which enables a simplified and reliable detection of leakage current.

The solution according to the invention resides in the features of the independent claim. The dependent claims relate to advantageous developments.

In the case of an electrosurgical generator configured to output a high-frequency AC voltage to an electrosurgical instrument, comprising an inverter for high voltage, which generates the high-frequency AC voltage that is passed via an output line to an output for connection of the electrosurgical instrument, and a leakage current detecting device for the electrosurgical instrument connected to the output, the invention provides for the leakage current detecting device to comprise a voltage measuring device, which is connected by its inputs in each case via a capacitive coupling to an active and a neutral line of the output line and has a bipolar voltage divider having a predetermined fixed division ratio, which has an upper connection and a lower connection, to which the capacitive coupling is applied, and also a center tap, and an asymmetry detector configured to compare an upper voltage between upper connection and center tap with a lower voltage between lower connection and center tap, and to output a fault signal for leakage current in the case of deviation of the ratio of upper voltage to lower voltage from the predetermined fixed division ratio.

The essence of the invention is the concept of carrying out, in the two lines of the output line, a measurement of the voltages prevailing there. This is done using the voltage divider, the division ratio of which is a predetermined fixed ratio, in the sense of a different voltage measurement. Two measurement signals are thus obtained, one, for each of the lines. They are fed to the asymmetry detector, which checks whether the two measured voltages are in the predetermined fixed ratio. If that is not the case, then that means that one of the two measured voltages deviates from its envisaged value, which may be caused by the occurrence of a leakage current in the affected line. The occurrence of leakage current is an indication that on this line there is a low-impedance path to ground (ground fault), for example owing to the fact that the patient to be operated on is touching a metal part that is grounded (by way of a protective conductor), as a result of which the potentially dangerous leakage current arises.

In this case, the invention makes use of the insight that the result of the voltage measurement with a bipolar voltage divider is shifted owing to the occurrence of a leakage current, and this shift is detected and signaled by the asymmetry detector. The signaling is a warning to the user (surgeon) of the electrosurgical generator and can furthermore function as a start signal for possibly automated rapid shutdown of the AC voltage that is output. This enables effective protection for the patient.

By virtue of the voltage measuring device having a bipolar voltage divider according to the invention, the measurement is a differential type of measurement and thus exhibits pronounced immunity to interference. Unlike in the prior art, it can thus be arranged anywhere on the output line and need not be arranged directly at the output. Complex measuring transducers, such as current transformers operating according to the transformer principle, are not required. This yields more design freedoms in terms of positioning and simplifies the construction of the electrosurgical generator and also the integration of the leakage current detecting device according to the invention. As a result, it is more robust and more reliable than previously customary methods for detecting a leakage current, which further improves patient protection.

An explanation is given below of a few terms that are used:

A voltage measuring device having a bipolar voltage divider is understood to mean such a voltage measuring device which measures two voltages in the same way. It is ideally constructed symmetrically in relation to the components that define the measurement, such that identical ratios in regard to measurement gain and frequency response are present. A special case thereof is a symmetrical voltage divider having identical impedances on both sides. A symmetrical voltage divider is not obligatory, in the present case, it is sufficient if at least the impedances of the voltage divider are in a predetermined fixed ratio to one another (as determined by the desired division ratio of the voltage divider). The impedances which are concerned here are in particular specifically those impedances which are arranged between upper connection and center tap as well as between center tap and lower connection. The fixed ratio is defined by the ratio (e.g. 2:1) of the impedances of the bipolar voltage divider. If the impedances are equal in magnitude, i.e. if the voltage divider is symmetrical, then the ratio is 1:1. This special case when the bipolar voltage divider is a symmetrical voltage divider provides for a circuit construction with a particularly clear layout and is therefore a preferred embodiment.

The comparison effected by the asymmetry detector is typically a comparison of the voltage level (amplitude and/or root-mean-square value), where the comparison should be understood broadly in the sense of checking not just for equality, but possibly for the presence of the predetermined fixed ratio. There is asymmetry in this sense if equality or the predetermined fixed ratio is not present. The asymmetry detector is configured to check this (is equality or the predetermined fixed ratio present).

In the field of electrosurgical generators, “high-frequency” is understood to mean frequencies typically in the range of 200 kHz to 4000 kHz. “High-voltage” is typically understood to mean voltages of up to 10 kV, preferably up to 5000 V.

The power provided by the electrosurgical generator is typically in the range of between 1 and 500 watts, wherein the load impedance may vary greatly, and output voltage and power output may accordingly likewise change greatly and quickly.

Expediently, the asymmetry detector has a minimum threshold, below which a fault signal is not yet output. The fault signal is output only if the minimum threshold is exceeded. A certain tolerance threshold can thus be defined, such that the fault signal is not already triggered in the event of tiny deviations such as may occur when there is a leakage current that is still low and not dangerous. An unnecessarily early shutdown is thus prevented.

Advantageously, the minimum threshold is adjustable, preferably depending on an operating mode of the electrosurgical generator and/or the connected instrument. This makes use of the insight that different operating modes of the electrosurgical generator, in particular different programmed modes, as they are called, signify a risk of differing magnitude for the patient. In this regard, in the case of clocked modes with a large duty cycle, high pulse-like peak voltages are achieved, which constitute a potentially higher threat and therefore necessitate a lower setting of the minimum threshold. A correspondingly more generous setting can be effected for other modes. The same applies, mutatis mutandis, to electrosurgical instruments of varying configuration, where the different design of the electrosurgical instrument and thus the risk affecting the patient can additionally be taken into account when determining the minimum threshold. In particular, the minimum threshold can be defined in an instrument-dependent manner, preferably in a memory which is assigned to the instrument and in which a value for the minimum threshold is stored. The memory can be provided on the instrument or in the electrosurgical generator.

The output line often comprises at its output a connection for an active electrode and a neutral electrode. The neutral electrode is typically provided for being connected to the patient over a large area, depending on the application. In this case, it is expedient if different minimum thresholds are adjustable for the active electrode leading to the instrument and the large-area neutral electrode leading to the patient.

That is preferably made possible by the setting of different minimum thresholds in the case of the voltage measuring device according to the invention.

In accordance with a further particular advantage of the invention, the asymmetry detector is furthermore provided with a polarity detector for the leakage current. In this way, it is possible to identify on which of the two lines of the output line the leakage current occurs. In particular, it is thus possible to rapidly signal whether the leakage current occurs at the active electrode or at the neutral electrode, which makes it easier for the surgeon to initiate corresponding countermeasures. Preferably, for this purpose, the asymmetry detector interacts with a display device, which correspondingly signals whether the leakage current occurs at the active electrode or the neutral electrode.

One expedient embodiment for the asymmetry detector has a comparator having two inputs, the upper connection being connected to one of the inputs and the lower connection being connected to the other input. The comparator enables the asymmetry to be detected in a reliable and not very complex manner. In particular, there is the possibility of embodying the comparator using analog technology. This affords the advantage of rapid processing and high reliability. This holds true particularly if the comparator is realized by means of an operational amplifier. A further expedient realization of the asymmetry detector is if the latter has a difference calculating unit with a threshold value switch, in particular a Schmitt trigger, connected downstream.

Preferably, the asymmetry detector has an analog/digital converter (ADC). This affords the advantage of digital signal processing. This provides advantages with respect to interference resistance as opposed to analog signals (being particularly important with respect to weak, small amplitude signals like those for leakage currents) and thereby increases measurement precision at one hand and flexibility with respect to arrangement of subsequent signal processing units at the other hand, due to those now being supplied with interference proof digital signals. This increased flexibility is applicable in particular with respect to structural arrangement within the electrosurgical generator, which is a significant practical advantage in consideration of confined spaces at the output outlets. Compromising between (analog) signal quality and structural arrangement which was required hitherto is thus no longer necessary due to the high (digital) signal quality being maintained independent of structural arrangement.

Preferred is in particular an arrangement of the analog/digital converter (ADC) on the input side of the asymmetry detector. Particularly preferred is an arrangement direct at the upper as well as lower connection, if applicable after pre-amplification, thereby achieving a conversion to digital signals rather early along the signal path. To this end, expediently two analog/digital converters are provided or a double analog/digital converter. This brings the advantage that the transition to interference free digital signals already have happens immediately subsequent to generating the signals for the leakage current. Preferably this is performed prior to asymmetry detection, thereby enabling an analyzing of the asymmetry already in the digital domain. Thereby the advantages of digital signal processing can be realized already for asymmetry detection.

Further, an analog/digital conversion as early as possible in the signal chain brings the advantage of allowing implementation of advanced analysis functions, in particular spectral analysis or analysis in the frequency domain, be it in the asymmetry detector and/or in other subsequent units for signal processing. By such spectral analyzes in particular higher harmonic frequency components can be evaluated. To this end the asymmetry detector can be configured for advanced analysis functions, in particular spectral analysis or analysis in the frequency domain. This provides for an improved evaluation and therefore detection precision, and on the other hand also increases flexibility with respect to different kinds of electrosurgical instruments.

Flexibility with respect to different kinds of electrosurgical instruments (including those which will become available only in the future, after the respective electrosurgical generator has been manufactured) can be increased by providing different thresholds dependent on the electrosurgical instrument which can be taken into consideration in an especially simple and preferably manner owing to digital signal processing. This can be accomplished e.g. by means of instrument individual threshold values which are correspondingly stored in the electrosurgical generator or by means of corresponding minimum thresholds being stored as a value in a memory of the respective instrument. Thereby an analog/digital converter enables a simplified implementation of different minimum thresholds, also with respect to future instruments. Thereby flexibility as well as future proofing of the electrosurgical generators are increased.

Advantageously, the capacitive coupling is embodied at high impedance relative to the voltage divider, preferably by at least a factor of 100, in particular at least 1000. Expediently, the capacitive coupling has capacitors in the Picofarad range, preferably at most 20 Picofarads. Such a high impedance capacitive coupling further provides the advantage that the voltage divider which is positioned—viewed from the active and neutral line—behind the coupling is “high-voltage free”, i.e. is not subjected to high voltage. Therefore no high-voltage robust components (particular capacitors) are required. This is a significant practical advantage since often a conflict of aims arises between high-voltage robustness at one hand and close-tolerance, precise determination of the division ratio at the other hand.

Expediently, the voltage divider is embodied in a capacitive fashion. This avoids the influence of DC current and enables a favorable frequency response behavior, in particular in interaction with the capacitive coupling. Moreover, compared with a resistive voltage divider, the capacitive embodiment provides for additional isolation, in particular vis a vis undesired DC current.

Advantageously, the center tap of the voltage divider is connected to a fixed potential. This creates a virtual ground for the voltage measurement, thus an unambiguous reference potential. In particular, provision can be made for connecting the center tap to a protective conductor and thus electrically to ground (protective ground or protective earth). That has the advantage that the center tap is thus connected to the same potential to which leakage currents possibly flow as well. Expediently, for this purpose, the ground of the measuring electronics is also grounded (protective ground or protective earth).

Furthermore, provision can be made for the leakage current detecting device to interact with a separate device for determining a magnitude of the current, in particular of the leakage current. It is primarily of importance, particularly for the safety of the patient, to rapidly and reliably detect that a leakage current is present. A secondary consideration is that there is a further advantage in being able to ascertain a measure of the magnitude of the leakage current in order thus to provide valuable additional information for the localization of the fault resulting in the leakage current. By way of example, such a device is configured to interact with the operational controller of the generator. On the basis of measurement values for the current in the output line that are generally present in the operational controller anyway, said device can compare with one another current measurement values of the current output overall directly before and after detection of the leakage current by the asymmetry detector, and can determine therefrom a measurement of the magnitude of the leakage current.

The invention is explained in greater detail below on the basis of an advantageous exemplary embodiment with reference to the accompanying drawing, in which:

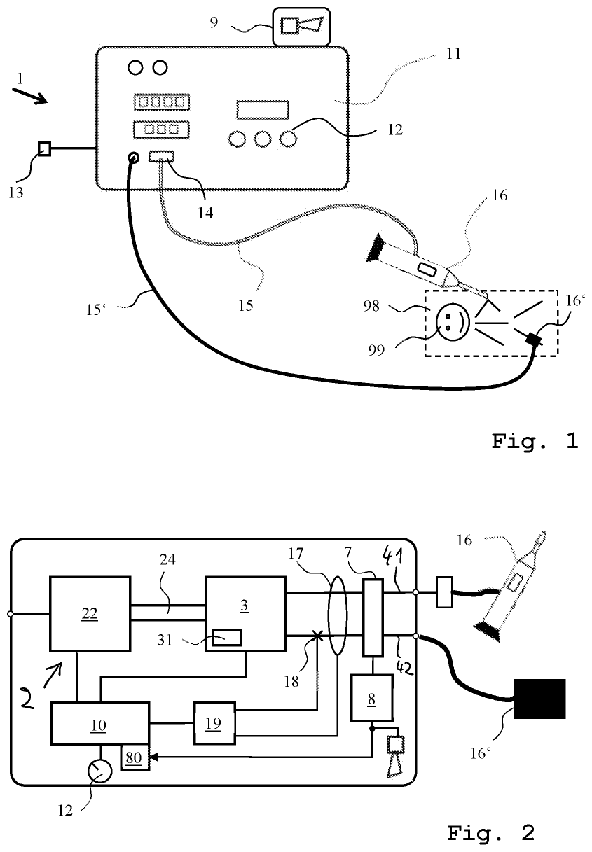

FIG. 1 shows a schematic illustration of an electrosurgical generator in accordance with one exemplary embodiment with a connected electrosurgical instrument;

FIG. 2 shows a block diagram concerning the electrosurgical generator in accordance with FIG. 1;

FIG. 3 shows an exemplary circuit diagram for a measurement sensor with a leakage current detecting device;

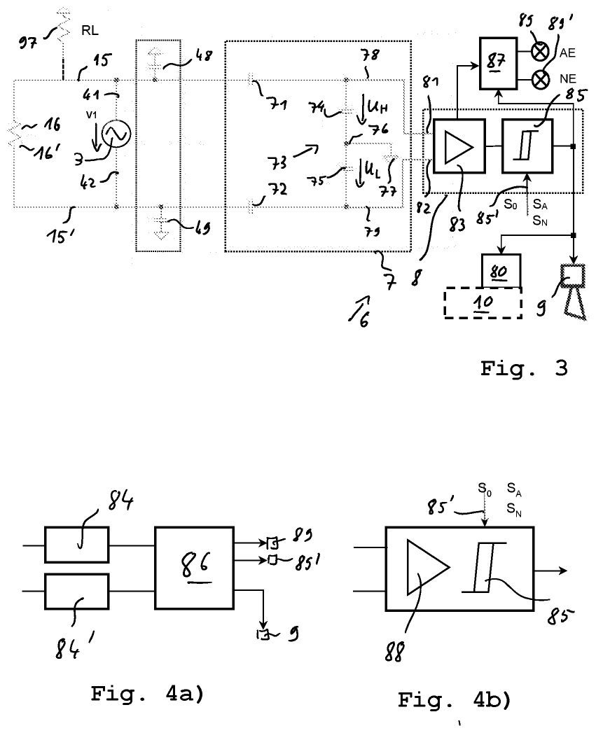

FIG. 4a, b show embodiment variants for an asymmetry detector with respect to FIG. 3;

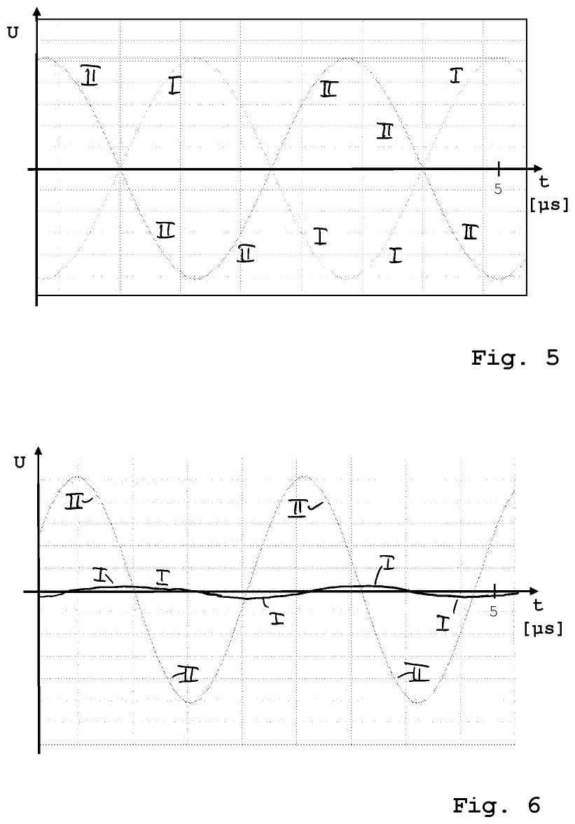

FIG. 5 shows a diagram concerning measured voltages without leakage current; and

FIG. 6 shows a diagram concerning measured voltages with leakage current.

An electrosurgical generator in accordance with one exemplary embodiment of the invention is illustrated in FIG. 1. The electrosurgical generator, designated in its entirety by the reference number 1, comprises a housing 11 provided with a connection 14 for an electrosurgical instrument 16. In the exemplary embodiment illustrated, the instrument is an electric scalpel. It is connected to the connection 14 of the electrosurgical generator 1 via a high-voltage connecting cable 15. A further connecting cable 15′ is led to an operating table 98 and connected there to a counter electrode 16′. The latter is configured to be arranged over a large area on the patient 99 to be operated on. The power output to the electrosurgical instrument 16 can be changed by way of a power controller 12.

For the following explanation of the construction of the electrosurgical generator 1, reference is made in particular to FIG. 2. For supplying the electrosurgical generator 1 with power, a DC voltage supply 2 is provided. It can be connected to the public electricity supply system via a supply system connection cable 13 and can be fed via a high-voltage power supply unit (High Voltage Power Supply—HVPS). The power supply unit 22 comprises a rectifier and feeds a DC voltage link circuit 24 in the exemplary embodiment illustrated. It should be noted that the supply from a power supply unit 22 is not mandatory, rather that other types of DC voltage supply 2 are also conceivable, for example a direct supply with DC voltage, particularly in the case of electrosurgical generators installed in vehicles or in the case of those provided in mobile or temporary hospitals.

The magnitude of the DC voltage is typically between 10 and approximately 500 volts, often 48 volts in the case of modern electrosurgical generators. It can be fixed or variable, which is dependent in particular on the design of the inverter that generates the high voltages. The absolute magnitude of the DC voltage may depend in particular on the power set, the type of electrosurgical instrument 16 and/or the load impedance thereof, which in turn depends on the type of tissue treated.

An inverter 3 is fed by the DC voltage supply 2 and generates from the DC voltage fed to it high-frequency AC voltage in the high-voltage range of a few kilovolts with frequencies in the range of between 200 kHz and 4 MHz. The inverter 3 provided can be a so-called single-ended converter, for example, which is driven by an oscillator in a free-running manner; these are generally supplied by a DC voltage supply 2 with variable voltage. This embodiment can afford the advantage of conceptional simplicity and generally directly passes the high voltage generated by it via an equipment-internal output line 4 to the output connection 14 for the electrosurgical instrument 16. Alternatively, however, provision can also be made for the inverter 3 to be embodied as an inverter. In the case of the latter, the setting of the power and also of the voltage to be output is effected by way of the inverter itself, such that a variable DC voltage supply 2 is not required, rather one with a fixed voltage (for example 48 V) is sufficient. The inverter has power semiconductor switches as so-called current valves, which are driven by an inverter controller 31 in a manner known per se, for example by means of known pulse width modulation as PWM control, for the purpose of generating a high-frequency high voltage. The high-frequency high voltage generated by the inverter is thus virtually freely adjustable with regard to frequency and waveform. The high-frequency voltage generated by the inverter is typically output via a low-pass filter and an output transformer (not illustrated) for voltage boosting to the generator-internal output line 4 to the connection 14 for the electrosurgical instrument 16.

Furthermore, voltage and current of the high voltage generated by the inverter 3 are measured by means of a voltage sensor 17 and a current sensor 18, and the measurement signals are fed to a processing unit 19, which applies the corresponding data about the output voltage, current and power to an operational controller 10 of the electrosurgical generator 1. The power controller 12 is also connected to the operational controller 10. The operational controller 10 is furthermore configured to set various modes, as they are called, which typically involve stored voltage/time profiles; however, they can also involve predefinitions containing the waveform of the high-frequency high voltage to be output.

The high-frequency high voltage generated is passed to the output connection 14 via the output line 4 with its lines 41 for an active electrode and line 42 for a neutral electrode. Owing to the high frequency of the voltage output by the electrosurgical generator, parasitic capacitances act on the lines 41, 42. They are represented by the capacitive elements 48, 49 in FIG. 3.

The electrosurgical instrument 16 is connected to the output connection 14. In the exemplary embodiment illustrated, this is a monopolar instrument connected to the line 41 with the active electrode; however, the use of bipolar instruments (not illustrated) can also be provided. In order to close the electric circuit, a counter electrode 16′ is connected to the line 42 via the connecting cable 15′. The counter electrode 16′ is situated on the operating table 98, the patient 99 to be operated on lying said table. There the counter electrode 16′ is connected to the patient 99 over a large area at a suitable location (this is illustrated merely symbolically in FIG. 1). Once the surgeon then places the electrosurgical instrument 16 on the patient 99, the electric circuit is closed via the body tissue of the patient 99. The body tissue situated directly at the instrument tip 16 is heated as a consequence of the contact resistance prevailing there and it is cut, cauterized, coagulated, etc., depending on the electrosurgical instrument 16 used.

It is critical if the electric circuit is (also) closed via other locations, in particular body parts of the patient 99. Uncontrolled leakage currents arise there. That is a considerable risk for the safety of the patient, possibly also of the medical personnel. In order to detect this, a leakage current detecting device 6 is provided.

For further explanation, reference is now made to FIG. 3, where the inverter 3 is symbolically illustrated as a source of the AC voltage in the left-hand part of the figure. Said voltage is output to the connecting cable 15 for the electrosurgical instrument 16 via the output line 4 with its lines 41, 42 for the active and neutral electrodes, respectively. The electric circuit is closed via the further connecting cable 15′. The electrosurgical instrument 16 and also the counter electrode 16′ are symbolically represented by a load resistor in FIG. 3.

The leakage current detecting device 6 comprises a bipolar voltage measuring device 7 having a bipolar voltage divider 73, and also an asymmetry detector 8. The voltage measuring device 7 is configured to carry out a voltage measurement on the lines 41, 42 of the output line 14 for the active and neutral electrodes. In the exemplary embodiment, the bipolar voltage divider of the voltage measuring device 7 is configured by way of example as a symmetrical voltage divider 73 having a division ratio of 1:1, which is embodied as a capacitive voltage divider having an upper measurement capacitance 74 and a lower measurement capacitor 77, which are connected to one another at a center tap 77. The respective other connection of the measurement capacitance 74, 77 is arranged at an upper connection 78 and a lower connection 79, respectively, of the voltage divider 73. The magnitude of the measurement capacitance is in the nanofarad range, for example approximately 10 nF.

For a measurement of the voltage in the lines 41, 42 that is as free of perturbations as possible, the symmetrical voltage divider 73 is connected to the lines 41, 42 via a high-impedance coupling. The high-impedance coupling is implemented by means of two capacitors having low capacitances, a capacitor 71 as connection between the line 41 and the upper connection 78 of the voltage divider 73 and also a second capacitor 72 as connection between the line 42 and the lower connection 79 of the voltage divider 73. The two capacitors 71, 72 have a capacitance in the Picofarad range, for example approximately 3 pF.

The upper connection 78 and the lower connection 79 are led out of the voltage divider 7 as output connections. They are applied to inputs 81, 82 of the asymmetry detector 8 connected downstream. The latter is configured to compare with one another the two voltages coming from the voltage divider 73 at the upper connection 78 and lower connection 79, respectively, and to check whether they have the predetermined fixed ratio (in the example, this is 1:1, since the voltage divider is symmetrical), i.e. have amplitudes (or root-mean-square values) of identical magnitudes. It should be noted that for the embodiment of the voltage measuring device 7 and of the asymmetry detector 8, it is not mandatory for both voltages to have exactly identical magnitudes, rather that this could also be implemented such that both voltages have a previously defined fixed ratio. Only of the case where both voltages are intended to be equal in magnitude is explained below, for the sake of simplification; the explanations apply, mutatis, mutandis, to other predefined fixed ratios. Inputs 81 and 82 of the asymmetry detector 8 are connected to the positive and negative inputs, respectively, of a comparator 83. The latter is configured to compare the two applied voltages with regard to their magnitude and to output a corresponding output signal which signals whether or not one voltage is higher than the other. Depending on the embodiment of the comparator 83, this output signal can be proportional to the deviation or digital, that is to say that it only provides information about whether or not there is equality.

In the last-mentioned case, the output of the comparator 83 can function directly as a fault signal and be applied to a signaling device, for example to an acoustic signal generator in the form of a warning horn 9.

During regulation operation, the voltages output by the symmetrical voltage divider 73 at the upper connection 78 and lower connection 79 are equal in magnitude, as is also illustrated in FIG. 5, where the curve designated by I shows the profile of the voltage U_{H} present at the upper connection 78 and the curve II shows the profile of the voltage U_{L} present at the lower connection 79. In the example illustrated, the two voltages are equal in magnitude and correspond to half of the voltage V1 output by the inverter 3, provided that no fault situation is present and, in particular, there is no ground fault in the region of the lines 15, 15′ of the electrosurgical instrument 16 or on the patent 99 with the latter's support 98. The comparator 83 ascertains that the voltages are equal in magnitude and does not output a fault signal.

The situation is different if a fault situation is present, i.e. if for example one of the body parts of the patient 99 touches a grounded component (grounded metal part). A parasitic impedance 97 to ground is thus formed, via which a leakage current flows. The voltages at the upper connection 78 and lower connection 79 that are ascertained by the voltage measuring device 73 are thus no longer equal in magnitude. As illustrated in FIG. 6, the voltage in the line 41 and thus at the upper connection dips owing to the leakage current. The resulting voltage profile for U_{H} is represented by the curve I progressing close to the zero line. By contrast, the voltage U_{L} at the lower connection 79, represented by the curve II, is maintained. As is clearly evident in FIG. 6, when a leakage current occurs, the voltages are not equal in magnitude, but rather have considerable differences. This is ascertained by the comparator 83 of the asymmetry detector 8. It outputs a corresponding fault signal which is passed to the warning horn 9 and thus provides the surgeon with a readily perceptible warning signal for the presence of the leakage current. At the same time, the signal can be connected to the operational controller 10 of the electrosurgical generator 1, such that the latter can react to the detected fault situation, for example by means of a rapid shut down of the inverter 3.

If the intention is for a fault signal not already to be triggered in the case of tiny deviations, then it is possible to provide the Schmitt trigger 85 as a threshold value switch. The latter has an adjustable threshold value 85′. In this way, it is possible to set what magnitude is permitted for the difference between the voltages at the upper connection 8 and at the lower connection 79 before the fault signal is triggered.

Expediently, a polarity detector 87 is furthermore provided. The input thereof is connected to the comparator 83. The sign from the result of the comparator 83 can thus be used to ascertain whether the leakage current flows away from the upper line 41 with the active electrode or from the connection line 15 (so-called “AE” fault), or whether the leakage current flows away from the lower line 42 with the neutral electrode or the connection line 15′ (so-called “NE” fault). Depending on that the polarity detector 87 drives a signal luminaire 89, 89′, which correspondingly signals the presence of an AE or NE fault, respectively.

Alternative embodiment variants for the asymmetry detector 8 are indicated in FIGS. 4a) and b). In the case of the variant in accordance with FIG. 4a), two analog/digital converters 84, 84′ are provided. The upper connection 78 is connected to the analog/digital converter 84, such that this converter converts a voltage signal for the voltage of the active electrode in the connecting line 15 into a corresponding first digital signal. The lower connection 79 is connected to the analog/digital converter 84′, such that this converter converts a voltage signal for the voltage of the neutral electrode, such as is present in the line 15′, into a corresponding digital signal. These two digital signals are applied to the inputs of a difference element 86. The latter is embodied using digital technology, for example using microprocessor technology. A check is made to establish the magnitude of the difference between the two digitally converted voltage signals. If it exceeds a likewise digitally adjustable threshold value (not illustrated in FIG. 4a), then the outputs of the difference elements 86 are activated. The latter has an output for connection to the, preferably acoustic, signal device 9 and also two further outputs, to which displays 89, 89′ are connected, which are indicative of whether the leakage current is to be assigned to the active electrode “AE” or to the neutral electrode “NE”.

FIG. 4b) illustrates a simplified variant. In the case of the latter, the signals coming from the voltage divider 73 for the voltage at the upper connection 78 and the voltage at the lower connection 79 are applied to a Schmitt trigger 85 with an integrated difference forming unit 88, for example a comparator circuit. Here, too, as in the case of the Schmitt trigger 85 in FIG. 3, the switching threshold of the Schmitt trigger can be adjusted by way of an adjustable threshold value signal 85′.

In this way, with little additional outlay, the invention enables a reliable detection of whether a leakage current occurs. Furthermore, with little outlay, the invention can ascertain the polarity, i.e. whether said leakage current occurs at the active electrode “AE” or the neutral electrode “NE”.

In addition to the warning horn 9, the fault signal output by the asymmetry detector 8 can be applied to a separate device 80 that interacts with the operational controller 10. The device 80 is expediently configured, in the case where the asymmetry detector 8 detects the occurrence of leakage current, to ascertain the magnitude of the leakage current. This can be done for example by a procedure in which, by means of the operational controller 10, on the basis of measurement values for the current in the output line such as are present there anyway and originate from the measuring sensor 18, said device compares with one another current measurement values of the current output overall directly before and after detection of a leakage current by the asymmetry detector 8 and optionally outputs them via a display device.

1. An electrosurgical generator configured to output a high-frequency AC voltage to an electrosurgical instrument, comprising an inverter for high voltage, which generates the high-frequency AC voltage that is passed via an output line to an output for connection of the electrosurgical instrument, and a leakage current detecting device for the electrosurgical instrument connected to the output.
wherein
the leakage current detecting comprises
a voltage measuring device, which is connected by its inputs in each case via a capacitive coupling to an active and a neutral line of the output line and has a bipolar voltage divider having a predetermined fixed ratio, which has an upper connection and a lower connection, to which the capacitive coupling is applied, and also a center tap, and
an asymmetry detector configured to compare an upper voltage between upper connection and center tap with a lower voltage between lower connection and center tap, and to output a fault signal for leakage current in the case of deviation of the ratio of upper voltage to lower voltage from the predetermined fixed ratio.

2. The electrosurgical generator as claimed in claim 1, wherein the asymmetry detector has a minimum threshold, below which a fault signal is not yet output.

3. The electrosurgical generator as claimed in claim 2, wherein the minimum threshold is adjustable.

4. The electrosurgical generator as claimed in claim 2, wherein different minimum thresholds are provided for the active electrode and the neutral electrode.

5. The electrosurgical generator as claimed in claim 3, wherein the minimum threshold is defined in an instrument-dependent manner.

6. The electrosurgical generator as claimed in claim 1, wherein the asymmetry detector is provided with a polarity detector for the leakage current.

7. The electrosurgical generator as claimed in claim 6, wherein the polarity detector interacts with a display device, which signals whether a leakage current occurs at the active electrode or the neutral electrode.

8. The electrosurgical generator as claimed in claim 1, wherein the asymmetry detector has a comparator having two inputs, the upper connection being connected to one of the inputs and the lower connection being connected to the other input.

9. The electrosurgical generator as claimed in claim 8, wherein the comparator is embodied using analog technology.

10. The electrosurgical generator as claimed in claim 1, wherein the asymmetry detector is embodied as a difference calculating unit with a threshold value switch connected downstream.

11. The electrosurgical generator as claimed in claim 1, wherein the asymmetry detector has an analog/digital converter.

12. The electrosurgical generator as claimed in claim 11, wherein the analog/digital converter is arranged on the input side of the asymmetry detector.

13. The electrosurgical generator as claimed in claim 1, wherein coupling is at high impedance relative to the voltage divider.

14. The electrosurgical generator as claimed in claim 1, wherein the voltage divider is capacitive.

15. The electrosurgical generator as claimed in claim 1, wherein the center tap is connected to a fixed potential.

16. The electrosurgical generator as claimed in claim 1, wherein the leakage current detector interacts with a device for determining a magnitude of the current.

17. The electrosurgical generator as claimed in claim 1, wherein the voltage divider is a symmetrical voltage divider.
